(19) United States
(12) Patent Application Publication
(10) Pub. No.: US 2021/0308319 A1
(43) Pub. Date: Oct. 7, 2021

(54) TRANSITION TO TRANSLUCENT SUTURES

(71) Applicant: University of Pittsburgh - Of the Commonwealth System of Higher Education, Pittsburgh, PA (US)

(72) Inventors: Eric J. Beckman, Pittsburgh, PA (US); Joseph P. Rubin, Pittsburgh, PA (US); Stephen D. Cummings, Pittsburgh, PA (US)

(73) Assignee: University of Pittsburgh - Of the Commonwealth System of Higher Education, Pittsburgh, PA (US)

(21) Appl. No.: 17/265,490

(22) PCT Filed: Aug. 1, 2019
(86) PCT No.: PCT/US2019/044670

§ 371 Date: Feb. 2, 2021

Related U.S. Application Data
(60) Provisional application No. 62/714,510, filed on Aug. 3, 2018.

(51) Int. Cl. A61L 17/14; A61L 17/12; C08L 67/04; C08K 5/00

(52) U.S. Cl. CPC A61L 17/145; C08K 5/0041; C08L 67/04; A61L 17/12

(57) ABSTRACT

An absorbable medical article comprising:
an absorbable polymeric substrate; and
an absorbable composition disposed on at least a portion of a surface of the substrate, wherein the absorbable composition comprises:
(i) at least one absorbable coating polymer distinct from the substrate and (ii) a colorant that is miscible with the absorbable coating polymer.

This application claims the benefit of U.S. Provisional Application No. 62/714,510, filed Aug. 3, 2018, which is incorporated herein by reference.

### BACKGROUND

Dyed resorbable sutures are used in many procedures as they provide the surgeon with a clear view of the material during use. Clear sutures are typically employed in situations where the repair is made just under the skin, where visible sutures lines would be less than ideal for the patient during the several months required for the material to resorb. The clear sutures are preferred for these procedures despite the difficulty in seeing the material and the concomitant eye strain experienced by the surgeon.

### SUMMARY

Disclosed herein is an absorbable medical article comprising:

an absorbable polymeric substrate; and

an absorbable composition disposed on at least a portion of a surface of the substrate, wherein the absorbable composition comprises:

(i) at least one absorbable coating polymer distinct from the substrate and (ii) a colorant that is miscible with the absorbable coating polymer.

The foregoing will become more apparent from the following detailed description, which proceeds with reference to the accompanying figures.

Also disclosed herein is a method comprising:

applying the article of any one of claims 1 to 17 to a subject wherein the article is colored at the time of applying but then transitions to translucent within 1 hour to 28 days after the time of applying.

### BRIEF DESCRIPTION OF THE DRAWINGS

FIG. 1. The UV-Vis spectra of crystal violet over time from 0 to 10 minutes.

FIG. 2. The UV-Vis spectra of violet stearate over time from 0 to 10 minutes.

FIG. 3. The UV-Vis spectra of crystal violet and violet stearate dyes at 10 mins after addition of water.

FIGS. 4A-4C. Schematic cross-sections of a conventional bulk-dyed monofilament (FIG. 4A); a convention clear monofilament suture (FIG. 4B); and a suture with a dyed coating according to an embodiment of the present invention (FIG. 4C).

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

The term “absorbable” is meant to be a generic term, which may also include bioabsorbable, resorbable, bioresorbable, degradable, biodegradable, dissolvable or biodissolvable.

The term “aliphatic” is defined as including alkyl, alkenyl, alkynyl, halogenated alkyl and cycloalkyl groups. A “lower aliphatic” group is a branched or unbranched aliphatic group having from 1 to 10 carbon atoms.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

A “biodegradable polymer” is a polymer that degrades fully (i.e., down to monomeric species) under physiological conditions, meaning a temperature of 40° C. and below, pH of 6-9, and an aqueous solution.

The term “color” is inclusive of black and white. A “colored’ construct preferably absorbs light in a visible light region. The “visible light region” herein refers to a wavelength band between 360 nm and 830 nm or UV region between 120 and 360 nm.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

The term “translucent” describes the final, non-colored state of the polymeric substrate (e.g., a suture filament) after the coating polymer has been absorbed in vivo. The remaining translucent substrate that was underneath the coating polymer can be colorless (clear), or slightly off-white or beige. In either case, the substrate is “translucent”, with no colorant present after absorption of the colored coating polymer.

### Overview

Disclosed herein are colored constructs (e.g., medical articles) which are easily seen by a medical professional during a medical procedure, but that transition to a translucent state in vivo over a desired time period (e.g., 24 hours, 72 hours, 3 weeks) after the medical procedure. A substrate (e.g., a suture) is coated with an absorbable composition that includes (i) at least one absorbable coating polymer distinct from the substrate and (ii) a colorant (e.g., dye, color powder and/or pigment) that is miscible with the absorbable coating polymer, wherein the absorbable composition degrades faster than the substrate. Because the absorbable composition includes not only a miscible colorant, but also a coating polymer, the modified suture is sufficiently robust to be pulled through tissue without an initial loss of color (e.g., by avoiding removal of at least a portion of the colored coating via the friction derived from pulling the suture through tissue). Further, the use of a fast degrading coating polymer for the absorbable composition permits the disappearance of the color within a clinically relevant period of time. The coating polymer containing the colorant degrades in vivo, and the colorant and the polymer are dissolved by the body and excreted leaving the translucent substrate behind.

In certain embodiments, the colorant is not off-white. In certain embodiments, the colorant is not beige. In certain embodiments, the colorant is not white. In certain embodiments, the colorant is not black.

The colorant utilized herein may be a pigment or dye that has been modified to increase its hydrophobicity or hydrophilicity so that it will become miscible with a given coating polymer. A first approach for increasing hydrophobicity is to replace a dye or pigment inorganic counteranion with an organic alkanoate salt via an ion-exchange process. In the first approach, the dye is a positively charged dye such as crystal violet or methylene blue, and the inorganic anion of the dye is replaced with an alkanoate. A second approach for increasing hydrophobicity is to replace a dye or pigment inorganic countercation with quaternary phosphonium or ammonium via an ion-exchange process. In the second approach, the dye is a negatively charged dye such as Acid Green 25, and the inorganic cation of the dye is replaced with quaternary phosphonium or ammonium. One approach for increasing hydrophilicity would be to replace a dye or pigments inorganic counterion with a polyethylene oxide chain. The dye or pigment may be fully ion-exchanged or only partially ion-exchanged (e.g., up to 50%, up to 60%, up to 70%, up to 80%, or up to 90%).

The dye or pigment has an ionic character to enable the ion exchange. Illustrative dyes or pigments include crystal violet, methylene blue, acid green (e.g., acid green 25), thymol blue, chromium-cobalt-aluminum oxide, ferric ammonium citrate, pyrogallol, logwood extract, 1,4-bis[(2-hydroxy-ethyl)amino]-9,10-anthracenedione bis(2-propenoic)ester copolymer, 1,4-bis[(2-methylphenyl)amino]-9,10-anthracenedione, 1-bis[4-(2-methacryloxyethyl) phenylamino]anthraquinone copolymer, carbazole violet, chlorophyllin-copper complex (oil soluble), chromium oxide green, C.I. Vat Orange 1, 2-[[2,5-diethoxy−4-[(4-methylphenyl)thiol]phenyl]azo]-1,3,5-benzenetriol, 16,23-dihydrodinaphtho[2,3-a:2′,3′-i]naphth[2′,3′:6,7]indolo[2,3-c]carbazole-5,10,15,17,22,24-hexone, n,n′-(9,10-dihydro-9,10-dioxo-1,5-anthracenediyl)bisbenzamide, 7,16-dichloro-6,15-dihydro-5,9,14,18-anthrazinetetrone, 16,17-dimethoxydinaphtho (1,2,3-cd:3′,2′,1′-Im) perylene-5,10-dione, poly(hydroxyethyl methacrylate)-dye copolymer, Reactive Black 5, Reactive Blue 21, Reactive Orange 78, Reactive Yellow 15, Reactive Blue No. 19, Reactive Blue No. 4, C.I. Reactive Red 11, C.I. Reactive Yellow 86, C.I. Reactive Blue 163, C.I. Reactive Red 180, 4-[(2,4-dimethylphenyl)azo]-2,4-dihydro-5-methyl-phenyl-3H-pyrazol-3-one, 6-ethoxy-2-(6-ethoxy-3-oxobenzo[b]thien-2(3h)-ylidene) benzo[b]thiophen-3(2h)-one, phthalocyanine green, iron oxide, titanium dioxide, vinyl alcohol/methyl methacrylate-dye reaction product, C.I. Reactive Red 180, C.I. Reactive Black 5, C.I. Reactive Orange 78, C.I. Reactive Yellow 15, C.I. Reactive Blue No. 19, C.I. Reactive Blue 21, mica-based pearlescent pigment, disodium 1-amino-4-[[4-[(2-bromo-1-oxoallyl)amino]-2-sulphonatophenyl]amino]-9,10-dihydro-9,10-dioxoanthracene-2-sulphonate (Reactive Blue 69), D&C Blue No. 9, D&C Green No. 5, [phthalocyaninato(2-)]copper, FD&C Blue No. 2, D&C Blue No. 6, D&C Green No. 6, D&C Red No. 17, D&C Violet No. 2, D&C Yellow No. 10. In certain embodiments the dyes or pigments are FDA-approved for use in medical articles. In certain embodiments the dyes or pigments are those used in the operating room or medical research.

Illustrative alkanoates include those with C₁ to C₃₀ chains such as butanoate, octanoate, stearate, oleate, behenate, linoleate, arachadonate, myristoleate, palmitoleate, vaccenate, paullinate, eladate, eicosenoate, euricate, bassidate, nervonate, sapienate, gadoleate and petroselinate. Other polyunsaturated alkanoates can also be used. Illustrative cations for the alkanoate salt include Na⁺, K⁺, Li⁺ and Rb⁺.

Illustrative alkyl phosphoniums include those which are tetra-functionalized with C₁ to C₃O chains, and an inorganic counterion (like chloride, bromide or iodide) including tetradecyltrihexylphosphonium chloride, tetradecyltrihexylphosphonium bromide, tetrahexylphosphonium chloride, tetrahexylphosphonium bromide, tetraoctylphosphonium chloride, tetraoctylphosphonium bromide, tetrabutylphosphonium chloride, and Tetrabutylphosphonium bromide.

Illustrative alkyl ammoniums include those which are tetra-functionalized with C₁ to C₃₀ chains, and an inorganic counterion (like chloride, bromide or iodide) including tetraethylammonium chloride, tetraethylammonium bromide, tetrabutylammonium chloride, tetrabutylammonium bromide, tetrahexylammonium chloride, tetrahexylammonium bromide, tetraoctylammonium chloride, and tetraoctylammonium bromide

As illustrative examples, crystal violet and methylene blue were selected as dyes since the organic dye is a cation and the associated anion is an inorganic halide, chloride. Sodium butanoate, sodium octanoate, and sodium stearate were selected as hydrophobic counterions to replace the chloride counterions in crystal violet and methylene blue. The structures are shown below:

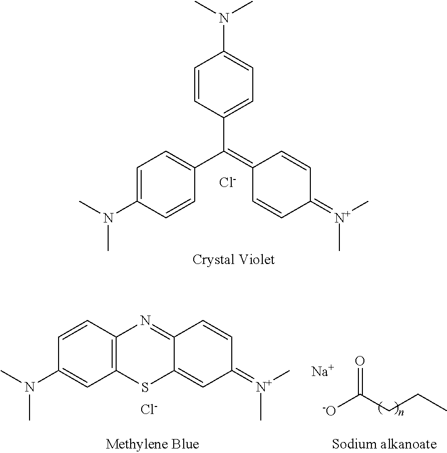

For the sodium alkanoates, n=4, 8, and 18.

The dyes were synthesized using the principle of salt metathesis, whereby a pair of counterions is precipitated or removed from the reaction driving the exchange equilibrium in favor of the desired salt. In this case, the desired salt is one composed of the dye cation and the alkanoate anion, with sodium chloride being removed from solution during the reaction. It follows then that a suitable solvent is necessary to dissolve the raw dye and sodium alkanoates, which also has a very low affinity for sodium chloride and will not dissolve more than a trace quantity of NaCl. The solvents chosen for these reactions were anhydrous ethanol and acetone. The same process may be used for ion exchange with an alkyl phosphonium.

The coating polymer for use in the composition is an absorbable polymer that degrades faster than the polymer(s) constituting the substrate. Illustrative absorbable polymers include aliphatic polyester, polyanhydride, aliphatic polyurethane, aliphatic polycarbonate, hydrophobically-modified polysaccharide, or a mixture thereof. The aliphatic polyester may be, for example, polylactic acid, polyglycolic acid, polycaprolactone, polydioxanone, or a copolymer of at least one monomer selected from lactic acid, glycolic acid, caprolactone, and/or dioxanone. The aliphatic polyurethane may be, for example, a polyurethane made from a lysine diisocyanate or other aliphatic diisocyanates and an aliphatic diol. The hydrophobically-modified polysaccharide may be, for example, butyl carboxycellulose. In certain embodiments, the absorbable polymer is poly(sebacic anhydride), which is an FDA-approved polymer that degrades faster than any other FDA-approved polymer.

The amount of colorant mixed with the absorbable coating polymer may vary. In certain embodiments the weight ratio of colorant to absorbable polymer may range from 1:10000 to 1:3, more particularly 1:1000 to 1:49.

In certain embodiments, the colorant is blended with the absorbable polymer at a temperature above the softening point of the absorbable polymer resulting in a liquid mixture. The liquid mixture is then applied to a surface of the substrate, for example, via coating, spraying, coextrusion, lamination or crystallization to form a layer on the surface of the substrate. The layer may cover all or only a portion of the surface of the substrate. The absorbable composition forms a layer of 2 mm to 10 nm, more particularly 200 lam to 1 μm, thickness on the surface of the substrate.

The polymeric material for the substrate may be any absorbable polymer, copolymer, or mixture thereof, made from, for example, glycolide, L(−)-lactide, D(+)-lactide, meso-lactide, epsilon-caprolactone, p-dioxanone, or trimethylene carbonate.

One application of absorbable polyesters is their use as surgical sutures. Absorbable sutures generally come in two basic forms, multifilament braids and monofilament fibers. For a polymer to function as a monofilament, it must generally possess a glass transition temperature, T_{g}, below room temperature. A low T_{g} helps to insure a low Young's modulus which in turn leads to filaments that are soft and pliable. A high T_{g} material would result in a wire-like fiber that would lead to relatively difficult handling sutures; in this art such sutures would be referred to or described as having a poor “hand”. If a polymer possesses a high T_{g}, and it is to be made into a suture, it invariably must be a construction based on multifilament yarns; a good example of this is a braid construction. It is known that monofilament sutures may have advantages versus multifilament sutures. Advantages of monofilament structures include a lower surface area, with less tissue drag during insertion into the tissue, with possibly less tissue reaction. Other advantages include no wicking into interstices between filaments in which bacteria can move and locate. There is some thought that infectious fluids might more easily move along the length of a multifilament construction through the interstices; this of course cannot happen in monofilaments. Monofilament fiber is generally easier to manufacture as there are none of the braiding steps usually associated with multifilament yarns.

Absorbable monofilaments sutures have been made from poly(p-dioxanone) and other low T_{g} polymers. A very important aspect of any absorbable medical article is the length of time that its mechanical properties are retained in vivo. For example, in some surgical applications it is important to retain strength for a considerable length of time to allow the body the time necessary to heal while performing its desired function. Slowly healing situations include, for example, diabetic patients or bodily areas having poor blood supply. Absorbable long term sutures have been made from conventional polymers, primarily made from lactide. Examples include a braided suture made from a high-lactide, and lactide/glycolide copolymer. Those skilled in the art will appreciate that it is clear that monofilament and multifilament absorbable sutures exist and that short term and long term absorbable sutures exist. It is to be understood that these polymers would also be useful in the construction of fabrics such as surgical meshes. Additional medical articles medical articles may include, in addition to meshes, the following conventional articles meshes, tissue repair fabrics, suture anchors, stents, orthopedic implants, staples, tacks, fasteners, suture clips, etc.

The sutures may be used in a conventional manner in conventional surgical procedures, e.g., to approximate tissue or affix tissue to medical articles. Typically, after a patient is prepared for surgery in a conventional manner, including swabbing the outer skin with antimicrobial solutions and anesthetizing the patient, the surgeon will make the required incisions, and, after performing the required procedure proceed to approximate tissue using the long-term absorbable sutures of the present invention. In addition to tissue approximation, the sutures may be used to affix implanted medical articles to tissue in a conventional manner. After the incisions are approximated, and the procedure is completed, the patient is then moved to a recovery area. The long-term absorbable sutures of the present invention in the patient retain their strength in vivo for the required time to allow effective healing and recovery.

If desired, the medical articles may contain other conventional medically useful components and agents. The other components, additives or agents will be present to provide additional desired characteristics to the monofilament sutures and other medical articles including but not limited to antimicrobial characteristics, controlled drug elution, therapeutic aspects, radio-opacification, and enhanced osseointegration.

In general, therapeutic agents which may be administered via compositions of the invention include, without limitation, antiinfectives, such as antibiotics and antiviral agents; analgesics and analgesic combinations; anorexics; antihelmintics; antiarthritics; antiasthmatic agents; adhesion preventatives; anticonvulsants; antidepressants; antidiuretic agents; antidiarrheals; antihistamines; anti-inflammatory agents; antimigraine preparations; contraceptives; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators, including general coronary, peripheral and cerebral; central nervous system stimulants; cough and cold preparations, including decongestants; hormones, such as estradiol and other steroids, including corticosteroids; hypnotics; immunosuppressives; muscle relaxants; parasympatholytics; psychostimulants; sedatives; tranquilizers; naturally derived or genetically engineered proteins, polysaccharides, glycoproteins, or lipoproteins; oligonucleotides, antibodies, antigens, cholinergics, chemotherapeutics, hemostatics, clot dissolving agents, radioactive agents and cystostatics. Therapeutically effective dosages may be determined by in vitro or in vivo methods. For each particular additive, individual determinations may be made to determine the optimal dosage required. The determination of effective dosage levels to achieve the desired result will be within the realm of one skilled in the art. The release rate of the additives may also be varied within the realm of one skilled in the art to determine an advantageous profile, depending on the therapeutic conditions to be treated.

Examples of antimicrobial agents useful with the sutures of the present invention include the polychlorophenoxy phenols such as 5-chloro-2-(2,4-dichlorophenoxy)phenol (also known as Triclosan). Examples of radio-opacification agents include barium sulfate while examples of osseointegration agents include tricalcium phosphate.

Suitable glasses or ceramics that may be incorporated into the medical articles of the present invention include, but are not limited to phosphates such as hydroxyapatite, substituted apatites, tetracalcium phosphate, alpha- and beta-tricalcium phosphate, octacalcium phosphate, brushite, monetite, metaphosphates, pyrophosphates, phosphate glasses, carbonates, sulfates and oxides of calcium and magnesium, and combinations thereof.

Modern surgical sutures generally range from Size 5 (heavy braided suture for orthopedics) to Size 11/0 (for example, a fine monofilament suture for ophthalmics). The actual diameter of thread for a given U.S.P. size differs depending on the suture material class. The diameters of sutures in the synthetic absorbable suture class are listed in the United States Pharmacopeia (USP) as well as in the European Pharmacopoeia. The USP standard is more commonly used. The novel sutures of the present invention can be made in a variety of sizes, including conventional suture sizes. The suture sizes of the monofilament sutures of the present invention will range from 10-0 to 5. The monofilament fibers of the present invention when used for constructing other medical articles such as meshes, etc., will typically have diameters in the range of about 1 to about 8 mils. Multifilament sutures when constructed from the fibers of the present invention will have a sufficiently effective denier per filament (dpf) to provide the desired properties, typically a dpf of about 0.5 to about 5.

The novel sutures of the present invention may be packaged in conventional suture packaging including polymer tray with tracks, paper folders, etc., with a polymer and/or foil overwrap that is hermetically sealed and impervious to moisture and microbes. The sutures will be sterilized preferably in their packages using conventional medical article sterilizations processes, such as ethylene oxide, radiation, autoclaving, etc. Those skilled in the art will understand that the optimal sterilization process chosen will not adversely affect the characteristics of the absorbable polymeric sutures.

The novel absorbable sutures of the present invention may be useful as monofilament surgical sutures. However, the filaments may be used in other conventional medical articles including, but not limited to, fibrous articles such as multifilament-based sutures and surgical fabrics including meshes, woven fabrics, nonwoven fabrics, knitted fabrics, fibrous bundles, cords, tissue engineering substrates, and the like. The surgical meshes may be made using conventional methods including knitting, weaving, air-laying, etc.

Medical articles made from the novel constructs of the present invention may be used in conventional surgical procedures using conventional surgical techniques. For example, surgical sutures that are mounted to conventional surgical sutures may be used to suture wounds, repair blood vessels and organs, close incisions, attach medical articles to tissue, etc. In the case of repairing wounds or closing incisions by approximating tissue edges about a wound or incision, the needle and suture are passed through tissue about the wound or incision one or more times, and the sides of the wound are brought together by tensioning the suture and securing it in place in a conventional manner such as with knots.

An illustrative substrate that may be utilized in the present invention include VICRYL® (polyglactin 910) Suture (commercially available from Ethicon), a synthetic absorbable sterile surgical suture composed of a copolymer made from 90% glycolide and 10% L-lactide; MONOCRYL® Sutures, a monofilament synthetic absorbable surgical suture prepared from a copolymer of glycolide and epsilon-caprolactone; PDS® II Sutures prepared from the polyester, poly (p-dioxanone). In certain embodiments, the suture also includes at least one antibacterial agent, which may be included within the suture construct itself or in a coating on the suture. The antibacterial agent may be effective against, for example, Staphylococcus aureus, Staphylococcus epidermidis, and other bacteria (e.g., Coated VICRYL® Plus Antibacterial (polyglactin 910) Suture (commercially available from Ethicon)).

In certain embodiments, all of the components of the medical article are FDA-approved components.

### Examples

Typically, a nitrogen atmosphere was used to minimize the quantity of water present in the reaction mixture. 50 ml of dry ethanol was used to dissolve 0.8 g of crystal violet, and the subsequent mass of required sodium alkanoate was dissolved in 20-40 ml of dry ethanol. These solutions were then combined, still under nitrogen, stirred vigorously and heated to 70° C., and left 20 hours to mix. The reaction mixture was then allowed to cool to room temperature and stirring ceased. After cooling the mixture was vacuum filtered immediately to remove residual sodium chloride, and the ethanol removed under vacuum.

The synthesized hydrophobic dyes are increasingly waxier and less crystalline as the hydrocarbon mass of the alkanoate counterion increases. Un-modified crystal violet is a crystalline solid.

### Solubility of Hydrophobic Dyes:

Solubility analysis was conducted in 500 ml of distilled water, and at 25° C. 500 ml of water was added to an excess mass of each compound crystal violet, violet butanoate, violet octanoate, and violet stearate. A stir bar was added and the solutions stirred vigorously for 15 minutes, this aqueous phase was then filtered and transferred to a flask to remove the water under vacuum. Weighing the flask before adding the dye solution, and comparing the mass after removing all water, yielded the mass of dyes the successfully dissolved in 500 ml. The results are shown below:

### Crystal Violet, 4 g/L

### Violet Butanoate 2.2 g/L

### Violet Octanoate, 1.7 g/L

### Violet Stearate, 0.8 g/L

### UV-Vis Spectroscopy Experiments:

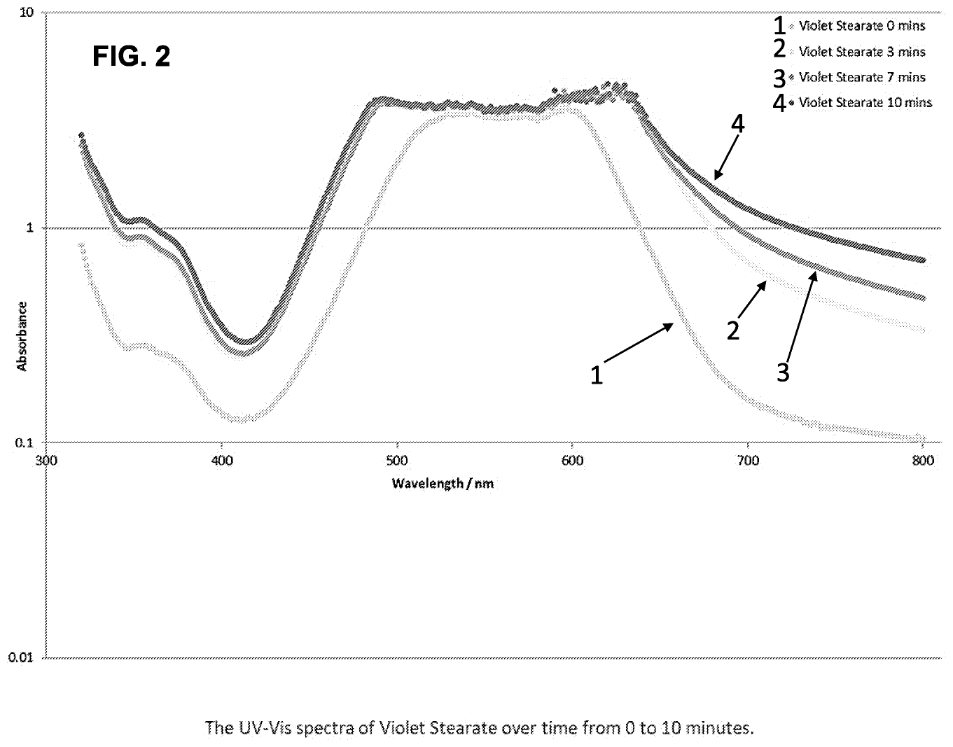

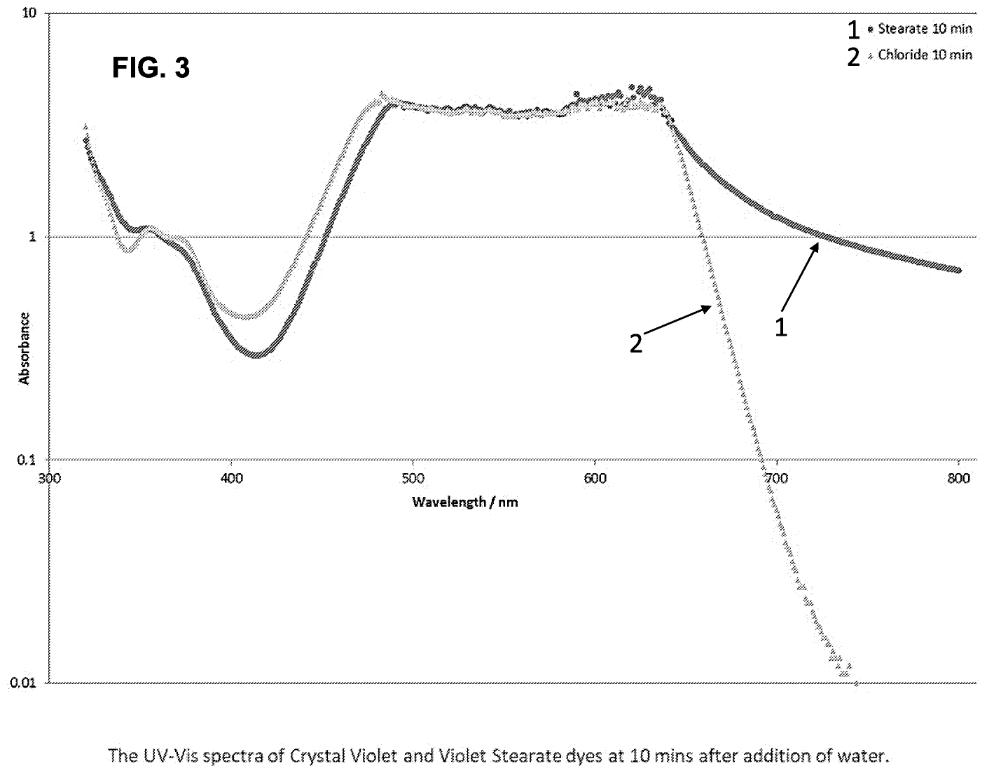

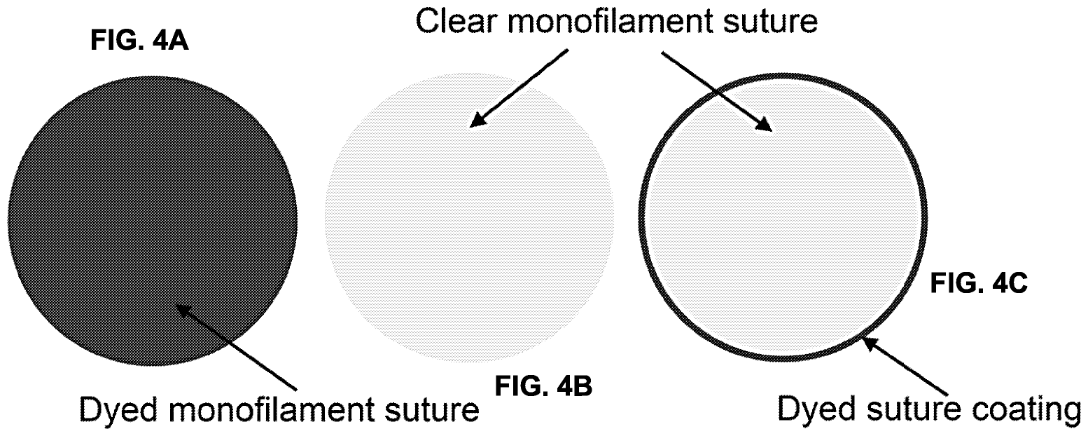

UV-Vis spectrums were done for the crystal violet and violet stearate dyes to measure any difference in rate of dissolution resultant from the different dye counterions, chloride and stearate. An excess mass of both crystal violet and violet stearate was added to separate, clean polystyrene cuvettes. The spectrometer was calibrated to measure absorbance over the range of 320-800 nm. A background UV-Vis spectrum was then collected using a clean cuvette filled with distilled water (this spectrum is later subtracted from the dye solution spectrums to give the absorbance resulting from the dye only). The dye containing cuvettes were then loaded into the UV-Vis spectrometer. Water was then carefully syringed down the side of the first cuvette until the cuvettes were filled, and the spectrum immediately collected upon addition. Absorbance spectrums were then recorded over time at 3 mins, 7 mins, 10 mins and 20 mins after the initial addition, for each dye. FIGS. 2, 3, and 4 (below) show the absorbance of crystal violet and violet stearate dyes at 10 mins dyes over time, as well as a comparison of the two dyes at 10 mins.

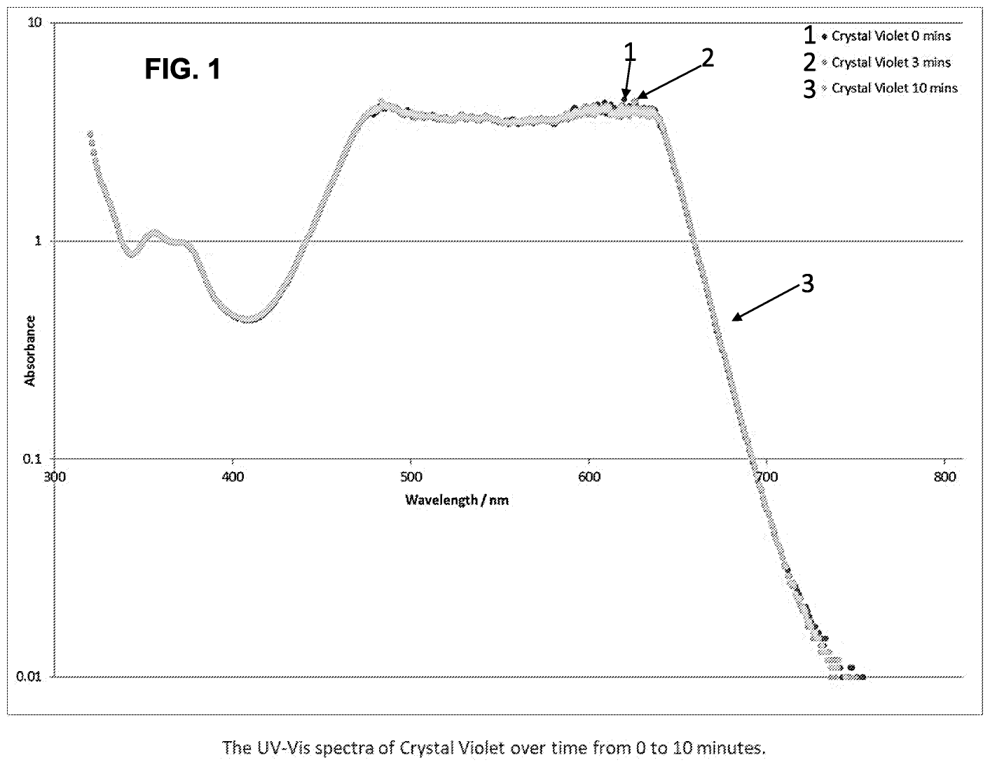

Analyzing FIG. 1, it can be seen that the crystal violet spectra do not change over time at all, all the spectrums overlay very closely. As can be seen from FIG. 2, violet stearate shows a definite increase in absorbance over time, for both short and long wavelengths. The 20 minutes spectra are not included due to the fact they are very similar to the 10 minute spectra for both dyes tested. Comparing both dyes at 10 minutes, the difference in absorbance can be seen between the crystal violet and the violet stearate.

In further examples, polycaprolactone (PCL) was combined with modified and unmodified pigments of methylene blue, and acid green 25. Methylene blue was modified to exchange its sodium form for butanoate. Acid green 25 was partially modified (only 50% of its sodium cation exchanged) with trihexyltetradecylphosphonium. PCL of 80,000 Mw was melted in a flask. (40,000 Mw was also tested but was less robust than 80,000 Mw, 14,000 Mw was found to too soft and flaked off of sutures after application with little force). Loadings were made of 0.5 wt % down to 0.01 wt % of dyes in the PCL melt. Undyed Biosyn 3-0 sutures (made by Covidien) were weighed and then coated with a fine layer of the pigmented polymer melt, and weighed again. To obtain a fine coating on the sutures, the sutures were dipped into the melt, then the needle pierced through two layers of foil to remove excess material. Coating loadings ranged from 0.1 g to 0.2 g. The pigment/dye faded completely after 3 weeks in vivo (rat experiments), leaving the color/transparency of the core polymer material of the suture itself prior to coating. A faster transition time may be achieved with a poly(sebacic anhydride) coating polymer and/or a thinner coating layer.

Additional examples employed the dyes acid green and thymol blue, commercially available polycaprolactone for the coating material, and conventional Vicryl® clear sutures for the substrate. Each dye was ion-exchanged (with a fatty acid for acid green, and an alkyl phosphonium for the thymol blue) and then blended with polycaprolactone above polycaprolactone's melting point of 60° C. Vicryl® sutures were then drawn through the caprolactone/dye liquid mixture and a lab-made die to create the coated suture.

Sutures were implanted in rats for 28 days; the animals showed no adverse medical issues over the full time of the experiment. Upon sacrifice, the animal tissue around the fade-to-clear sutures showed no retention of the dye, and the sutures had faded to the same clear tone as the control unmodified Vicryl® suture. The modified sutures were used to suture both rats and excised human tissue without any visible loss of the colored polycaprolactone layer. We employed

In view of the many possible embodiments to which the principles of the disclosed invention may be applied, it should be recognized that the illustrated embodiments are only preferred examples of the invention and should not be taken as limiting the scope of the invention.

1. An absorbable medical article comprising:
an absorbable polymeric substrate; and
an absorbable composition disposed on at least a portion of a surface of the substrate, wherein the absorbable composition comprises:
(i) at least one absorbable coating polymer distinct from the substrate and (ii) a colorant that is miscible with the absorbable coating polymer.

2. The article of claim 1, wherein the article is initially colored and then transitions to translucent within 1 hour to 28 days after in vivo use in a medical procedure on a subject.

3. The article of claim 1, wherein the absorbable composition forms a layer on at least a portion of a surface of the substrate.

4. The article of claim 1, wherein the absorbable polymeric substrate is a filament.

5. The article of claim 1, wherein the absorbable polymeric substrate is a monofilament or a multifilament, the monofilament or the multifilament having an outer surface, wherein the absorbable composition is disposed only on the outer surface.

6. The article of claim 5, wherein the absorbable composition coats all of the outer surface.

7. The article of claim 1, wherein the absorbable composition forms a layer of 2 mm to 10 nm thickness on the surface of the substrate.

8. The article of claim 1, wherein the colorant is crystal violet, methylene blue, acid green (e.g., acid green 25), thymol blue, chromium-cobalt-aluminum oxide, ferric ammonium citrate, pyrogallol, logwood extract, 1,4-bis[(2-hydroxy-ethyl)amino]-9,10-anthracenedione bis(2-propenoic)ester copolymer, 1,4-bis[(2-methylphenyl)amino]-9,10-anthracenedione, 1-bis[4-(2-methacryloxyethyl) phenylamino]anthraquinone copolymer, carbazole violet, chlorophyllin-copper complex (oil soluble), chromium oxide green, C.I. Vat Orange 1, 2-[[2,5-diethoxy-4-[(4-methylphenyl)thiol]phenyl]azo]-1,3,5-benzenetriol, 16,23-dihydrodinaphtho[2,3-a:2′,3′-i]naphth[2′,3′:6,7]indolo[2,3-c]carbazole-5,10,15,17,22,24-hexone, n,n′-(9,10-dihydro-9,10-dioxo-1,5-anthracenediyl) bisbenzamide, 7,16-dichloro-6,15-dihydro-5,9,14,18-anthrazinetetrone, 16,17-dimethoxydinaphtho (1,2,3-cd:3′,2′,1′-Im) perylene-5,10-dione, poly(hydroxyethyl methacrylate)-dye copolymer, Reactive Black 5, Reactive Blue 21, Reactive Orange 78, Reactive Yellow 15, Reactive Blue No. 19, Reactive Blue No. 4, C.I. Reactive Red 11, C.I. Reactive Yellow 86, C.I. Reactive Blue 163, C.I. Reactive Red 180, 4-[(2,4-dimethylphenyl)azo]-2,4-dihydro-5-methyl-phenyl-3H-pyrazol-3-one, 6-ethoxy-2-(6-ethoxy-3-oxobenzo[b]thien-2(3h)-ylidene) benzo[b]thiophen-3(2h)-one, phthalocyanine green, iron oxide, titanium dioxide, vinyl alcohol/methyl methacrylate-dye reaction product, C.I. Reactive Red 180, C.I. Reactive Black 5, C.I. Reactive Orange 78, C.I. Reactive Yellow 15, C.I. Reactive Blue No. 19, C.I. Reactive Blue 21, mica-based pearlescent pigment, disodium 1-amino-4-[[4-[(2-bromo-1-oxoallyl)amino]-2-sulphonatophenyl]amino]-9,10-dihydro-9,10-dioxoanthracene-2-sulphonate (Reactive Blue 69), D&C Blue No. 9, D&C Green No. 5, [phthalocyaninato(2-)] copper, FD&C Blue No. 2, D&C Blue No. 6, D&C Green No. 6, D&C Red No. 17, D&C Violet No. 2, D&C Yellow No. 10, or a mixture thereof.

9. The article of claim 1, wherein the colorant is an ion-exchanged dye or pigment.

10. The article of claim 1, wherein the colorant is an ion-exchanged dye having an organic alkanoate exchanged for a dye counteranion.

11. The article of claim 1, wherein the colorant is an ion-exchanged dye having a quaternary phosphonium or ammonium exchanged for a dye countercation.

12. The article of claim 1, wherein the colorant is an ion-exchanged dye having a polyethylene oxide chain exchanged for a dye counterion.

13. The article of claim 1, wherein the polymer of the absorbable polymeric substrate is a polymer, copolymer, or mixture thereof, made from glycolide, L(−)-lactide, D(+)-lactide, meso-lactide, epsilon-caprolactone, p-dioxanone, trimethylene carbonate, or a combination thereof.

14. The article of claim 1, wherein the absorbable coating polymer comprises aliphatic polyester, polyanhydride, aliphatic polyurethane, aliphatic polycarbonate, hydrophobically-modified polysaccharide, or a mixture thereof.

15. The article of claim 1, wherein the article is a monofilament, a monofilament suture, a multifilament suture, a surgical mesh, a surgical fabric, a film, a barbed suture, an injection molded article, or a combination thereof.

16. The article of claim 1, wherein the article is a suture.

17. The article of claim 1, wherein the absorbable composition degrades faster than the substrate in vivo.

18. A method comprising:
applying the article of claim 1 to a subject wherein the article is colored at the time of applying but then transitions to translucent within 1 hour to 28 days after the time of applying.
